(19) United States
(12) Patent Application Publication
(10) Pub. No.: US 2024/0305940 A1
(43) Pub. Date: Sep. 12, 2024

(54) SYSTEM AND METHOD FOR AUDITORY MIRROR THERAPY FOR HEARING DISORDERS

(71) Applicant: THE GENERAL HOSPITAL CORPORATION, Boston, MA (US)

(72) Inventor: Nils Clas Linnman, Peaks Island, ME (US)

(21) Appl. No.: 18/552,367

(22) PCT Filed: Mar. 16, 2022
(86) PCT No.: PCT/US2022/020609

§ 371 Date: Sep. 25, 2023

Related U.S. Application Data
(60) Provisional application No. 63/165,942, filed on Mar. 25, 2021.

(51) Int. Cl. H04R 25/00; A61M 21/00; H04R 1/10; H04R 5/027; H04R 5/033; H04R 5/04; H04S 7/00

(52) U.S. Cl. CPC H04R 25/75; A61M 21/00; H04R 1/1041; H04R 5/027; H04R 5/033; H04R 5/04; H04S 7/302; A61M 2021/0027; H04S 2400/11; H04S 2400/15

(57) ABSTRACT

Systems and methods for treating a subject with tinnitus are disclosed. The treatment can involve subjecting an individual afflicted with tinnitus to altered environmental sounds, wherein the altered environmental sounds are spatially altered forms of environmental sounds that the individual would naturally perceive. If the individual has a right ear and a left ear, a first audio sample from a location proximate to the left ear may be acquired along with a second audio sample proximate to the right ear. The treatment can then involve delivering at least a portion of the first audio sample to the right ear of the person experiencing tinnitus or delivering at least a portion of the second audio sample to the left ear of the person experiencing tinnitus, thereby creating at least a partial spatial transposition of the environmental sounds experienced by the individual.

### CROSS-REFERENCE TO RELATED APPLICATIONS

This application is based on and claims priority to U.S. Provisional Application Ser. No. 63/165,942, filed Mar. 25, 2021, and entitled, “SYSTEM AND METHOD FOR AUDITORY MIRROR THERAPY FOR HEARING DISORDERS.”

### BACKGROUND

Tinnitus is the perception of sound in the absence of any actual corresponding external sound. Chronic tinnitus is often the consequence of repeated high-intensity noise exposure and hearing loss, but can also result from ear infections and head or neck injuries. It is estimated that at least one in ten adults, or at least 30 million Americans, are currently bothered by tinnitus. Unfortunately, there are no current medications that reliably and substantially reduce the severity of tinnitus.

Treatments that have been somewhat effective in reducing tinnitus and tinnitus-related distress include cognitive behavioral therapy, hearing aids, and sound-masking therapies. Neuromodulatory approaches, including transcranial magnetic stimulation, transcranial direct current stimulation, vagus nerve stimulation, and deep brain stimulation have displayed only moderate success and are often costly, invasive, and not widely available.

Thus, there is a continued need for a safe, accessible, affordable, and non-invasive therapeutic approach that effectively treats chronic tinnitus.

### SUMMARY

The present disclosure overcomes the above and other drawbacks by providing systems and methods for treating an individual experiencing tinnitus. By altering the environmental sounds that an individual afflicted with tinnitus perceives, the symptoms experienced by the individual can be reduced. In some aspects, the methods and systems involve subjecting the individual to spatially altered forms of the environmental sounds that the individual would naturally perceive. For instance, an individual experiencing tinnitus may be effectively treated by transposing the sound delivered to the right ear to enter the left ear canal, and/or the sound delivered to the left ear to enter the right ear canal. The developed treatment techniques provide a safe, affordable, and noninvasive alternative to the therapeutic approaches currently available to treat tinnitus.

In accordance with one aspect of the disclosure, a method of treating tinnitus is provided. The method may include subjecting an individual afflicted with tinnitus to altered environmental sounds, wherein the altered environmental sounds are spatially altered forms of environmental sounds that the individual would naturally perceive.

In accordance with another aspect of the disclosure, a method of treating tinnitus is provided. The method can include acquiring a first audio sample from a location proximate to a left ear of an individual afflicted with tinnitus, acquiring a second audio sample from a location proximate to a right ear of the individual, delivering at least a portion of the first audio sample to the right ear of the individual, delivering at least a portion of the second audio sample to the left ear of the individual, and continuing delivery of the portion of the first audio sample delivered to the right ear of the individual or the portion of the second audio sample delivered to the left ear of the individual to carry out an effective therapy that reduces symptoms of tinnitus experienced by the individual.

In accordance with one aspect of the disclosure, a system for delivering a therapy for tinnitus is provided. The system may include a right microphone configured to sample environmental sounds from a location proximate to a right ear of an individual afflicted with tinnitus and a left microphone configured to sample environmental sounds from a location proximate to a left ear of the individual. The system may also include a right speaker configured to transmit sound to the right ear of the individual and a left speaker configured to transmit sound to the left ear of the individual. Furthermore, the system may include a controller configured to selectively control the right speaker to deliver the environmental sounds sampled by the left microphone to the right ear of the individual and selectively control the left speaker to deliver the environmental sounds sampled by the right microphone to the left ear of the individual to reduce symptoms of tinnitus experienced by the person afflicted with tinnitus.

In accordance with another aspect of the disclosure, a non-transitory, computer-readable storage medium is provided. The non-transitory, computer-readable storage medium may have instructions stored thereon that, when executed by a processor, cause the processor to carry out one or more steps. The one or more steps may include controlling a right microphone to sample environmental sounds from a location proximate to a right ear of an individual afflicted with tinnitus, controlling a left microphone to sample environmental sounds from a location proximate to a left ear of the individual, and selectively controlling a right speaker configured to be coupled to the right ear of the individual to deliver the environmental sounds sampled by the left microphone to the right ear or selectively controlling a left speaker configured to be coupled to the left ear of the individual to deliver the environmental sounds sampled by the right microphone to the left ear to reduce symptoms of tinnitus experienced by the individual.

The foregoing and other advantages of the invention will appear from the following description. In the description, reference is made to the accompanying drawings that form a part hereof, and in which there is shown by way of illustration one or more preferred embodiments of the invention. Such embodiments do not necessarily represent the full scope of the invention, however, and reference is made therefore to the claims and herein for interpreting the scope of the invention.

### BRIEF DESCRIPTION OF THE DRAWINGS

FIG. 1 is a schematic illustration of a flowchart for a method of treating tinnitus, in accordance with one aspect of the present disclosure.

FIG. 2 is a schematic illustration of a flowchart for a method of treating tinnitus, in accordance with another aspect of the present disclosure.

FIG. 3A is a schematic illustration of an exemplary system for delivering a therapy for tinnitus, in accordance with one aspect of the present disclosure.

FIG. 3B is a schematic diagram of the various components of FIG. 3A interacting with a remote server.

FIG. 4A is a schematic illustration of an exemplary system for delivering a therapy for tinnitus, in accordance with another aspect of the present disclosure.

FIG. 4B is a schematic diagram of the various components of FIG. 4A interacting with a remote server.

FIG. 5 depicts an image of the modified experimental ear defender headphones of the experiment of Example 1.

FIG. 6 depicts graphical results of the experiment of Example 1, charting each study participant's change in Tinnitus Handicap Inventory (THI) value over the number of hours the device was used.

### DETAILED DESCRIPTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices, and methods relating to treating an individual experiencing tinnitus are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, the term “tinnitus” has its ordinary meaning in the art. For instance, a subject may be considered to be afflicted with tinnitus or experiencing tinnitus if they experience some amount of ringing or other noise in one or both ears, either consistently or infrequently.

As used herein, the term “Tinnitus Handicap Inventory” also abbreviated as “THI” has its ordinary meaning in the art. For instance, the Tinnitus Handicap Inventory value can refer to a measured value representative of the severity of a perceived tinnitus handicap in a subject. The THI value may range from 0-100 and be measured using a 25-item self-reported questionnaire, with a consistent question set and scoring system. See, e.g., Newman C W, Jacobson G P, Spitzer J B. Development of the tinnitus handicap inventory. Arch Otolaryngol. 1996; 122:143-8.

As used herein, the term “Tinnitus Reaction Questionnaire” also abbreviated as “TRQ” has its ordinary meaning in the art. For instance, the Tinnitus Reaction Questionnaire value can refer to a measured value representative of the severity of a perceived tinnitus handicap in a subject. The TRQ value may range from 0-104 and be measured using a 26 item questionnaire, with a consistent question set and scoring system. See, e.g., Wilson P H, Henry J, Bowen M, Haralambous G. Tinnitus reaction questionnaire: psychometric properties of a measure of distress associated with tinnitus. J Speech Hear Res. 1991 February; 34(1):197-201. PMID: 2008074.

As used herein, the term “Tinnitus Handicap Questionnaire” also abbreviated as “THQ” has its ordinary meaning in the art. For instance, the Tinnitus Handicap Questionnaire value can refer to a measured value representative of the severity of a perceived tinnitus handicap in a subject. The THQ value may range from 0-100 and be measured using a 27 item questionnaire, with a consistent question set and scoring system. See, e.g., The psychometric properties of a tinnitus handicap questionnaire. Kuk F K, Tyler R S, Russell D, Jordan H Ear Hear. 1990 December; 11(6):434-45.

As used herein, the term “individual” may refer to a mammalian subject. The mammalian subject may specifically be a human being.

Unlike prior treatments for tinnitus, some aspects of the present disclosure utilize a technique which involves subjecting an individual afflicted with tinnitus to spatially altered forms of the environmental sounds that the individual would otherwise naturally perceive. In some forms, at least a partial transposition of the sounds perceived by an individual may be used as an effective form of treatment. In other words, the technique can rely on a “mirroring” of auditory perception in some forms, in that sounds coming from the right are experienced as coming from the left, and vice versa. This sound transposition is often referred to as Auditory Mirror Therapy (AMT) herein. As detailed below, this technique is based, in part, on neuroscience mechanisms of predictive coding of perceptions and multisensory integration. As detailed in Example 1 below, a tinnitus treatment device, or AMT device, was tested and showed that the spatially altered sound techniques described herein can be effective in treating tinnitus. Although the teachings described herein are often described with respect to treating various forms of tinnitus, such as chronic tinnitus and acute sound-induced tinnitus, one of skill in the art would readily recognize additional applications for the developed methods and systems.

Without being bound by theory, it is contemplated that if auditory brain pathways are deprived of their usual sensory input, this lack of signal is compensated for centrally by an adaption in signal gain, and this could be the basis of tinnitus. This intuitive paradigm is supported by many observations, but hearing loss alone it is not sufficient to explain tinnitus. For instance, the level of gain on auditory input is heavily modulated by attention. Up to nine out of ten persons with normal hearing and without tinnitus report “hearing something” when exposed to (and attending to) a completely silent room. Attention increases the gain in our primary senses (i.e. one “expects to hear something”), sometimes to the point where we perceive a transient phantom sound. Similar to other sensory nerve damage conditions, such as neuropathic pain and phantom sensations after limb loss, there appears to be a sophisticated integrative mechanism that typically adapts well to changes in peripheral signals, but under some conditions these adaptations instead “get stuck” in an increased gain mode that, despite peripheral normalization, is not re-calibrated but continues to give rise to chronic (now “phantom”) percepts. This sensory deprivation results in a prediction error between decreased bottom-up input and top-down prediction. Phantom percepts, such as pain and chronic subjective tinnitus, are thought to arise as a consequence of maladaptive bottom-up or top-down compensation to this prediction error, leading to persistent salience.

Unrelated to prior tinnitus treatments, mirror box therapy, where mirrors are placed so as to create a visual illusion of a limb in amputees, exploits the brain's preference to prioritize visual feedback over sensory feedback concerning limb position and proprioception. The continuous binding of multisensory input to form coherent perceptions gets hijacked with false information, resulting in a heavily modulated or illusory perception (i.e. the subject perceives their lost limb to be back). It is contemplated that this increase in error-signals facilitates a re-learning or re-interpretation of phantom pain sensations, possibly by triggering neural plasticity.

The human brain continuously binds information obtained through sensory channels to form a coherent representation of the world. These pieces of information complement and confirm each other, thereby improving the reliability of our perception. This is true for proprioception, but perhaps even more so for sound localization, where vision is used to calibrate and confirm where sound arises from. For sound, this adaptation is done to such a degree that when the information is incongruent, such as the sound and motion discrepancy in ventriloquism, the previously learned pattern (i.e. voices typically come from moving lips) is increased in weight. This generates the illusion that the puppet speaking, rather than us perceiving the true source from the ventriloquist's immobile lips. Thus, it is apparent that there is a powerful endogenous mechanism that modulates auditory perception. The neural circuits involved in tinnitus overlap those involved in neuropathic pain, and the perception of tinnitus (or phantom limb pain) appear to develop with changes in multisensory brain pathways that combine information coming from different senses. In tinnitus patients, neuroimaging studies have indicated consistently altered connectivity of in the bilateral anterior insula, the inferior frontal gyrus, the parahippocampal area, the middle temporal gyrus, cerebellum, and thalamus. It is thought that tinnitus may be a pathological condition which tends to produce hyperattention towards something that should not be salient to us. In line with this theory, widespread alteration in nucleus accumbens (NAc) connectivity, with tinnitus loudness correlated to the strength between the NAc and the parahippocampal cortex have been reported.

Recent mirror therapy studies indicate that mirror therapy for phantom limb pain normalizes visually evoked responses in the primary somatosensory cortex, in correlation with reduced pain. Modulating multisensory integration by increasing perceptual multisensory error signals is effective in phantom limb pain. In light of these realizations, the present disclosure applies the underlying theory of mirror therapy in a new context as an efficient way to treat tinnitus. By utilizing Auditory Mirror Therapy to subject an individual afflicted with tinnitus to spatially altered forms of the environmental sounds that the individual would otherwise naturally perceive, the various aspects of the present disclosure have the potential to provide a new class of methods and systems for treating tinnitus.

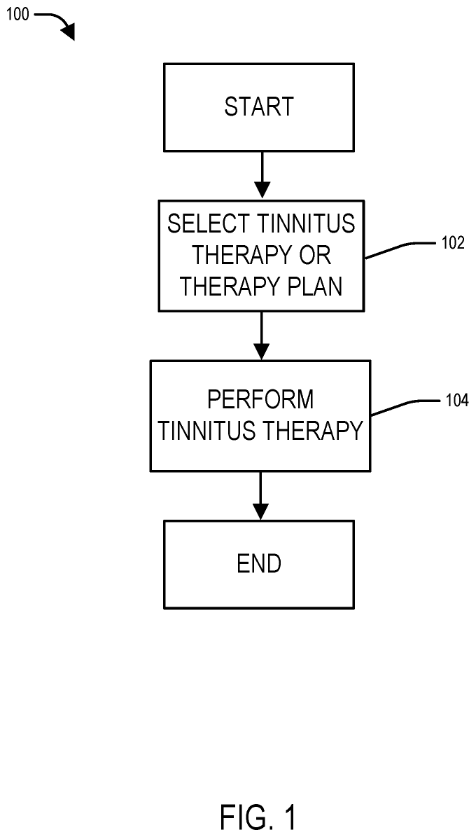

Referring to FIG. 1, a flowchart of an exemplary method of treating tinnitus 100 is provided. The method 100 includes selecting a therapy plan at step 102. For example, selection of a therapy plan may be achieved in a variety of settings. For example, a clinician may prescribe particular therapeutic parameters, such as particular parameters for altered environment sounds, durations of therapy, schedules for therapy, timing, frequency, volume or the like. As another non-limiting example, a user or patient may select a therapy, pre-configured therapy plan, or specific parameters for a therapy plan, including altered environmental sound parameters, including timing, duration, repetition, frequency, volume, and so on. As will be described, regardless of the party selecting the therapy or therapy plan, a therapy device may be used to then carry out the therapy or therapy plan at step 104. In particular, the therapy or therapy plan may include subjecting an individual afflicted with tinnitus to altered environmental sounds, wherein the altered environmental sounds are spatially altered forms of environmental sounds that the individual would naturally perceive.

The individual may have a right ear and a left ear and the method step of subjecting the individual to the altered environmental sounds may include acquiring a first audio sample from a location proximate to the left ear and acquiring a second audio sample proximate to the right ear. The first and second audio samples may be acquired using any suitable microphone or comparable device. The audio samples may be acquired at proximate locations and in a manner that allows the audio samples to substantially mimic the environmental sounds that would otherwise be acquired by that respective ear. For instance, if the full first audio sample was delivered to the left ear of the individual, they may not notice any difference between the delivered audio and the audio that the individual would otherwise anticipate based on other senses. However, the first and second audio samples need not necessarily be acquired proximate to the left ear or right ear of the individual. For instance, the sounds may be acquired at a different location on the head or body of the individual. For instance, the individual may be subjected to altered environmental sounds in their left ear that they would expect to originate from behind their head. Furthermore, the individual may be subjected to spatially altered sounds in only one ear. In the case were a subject suffers from tinnitus in only one ear, the altered environmental sounds may be specifically provided to only that afflicted ear, and the other ear may be allowed to receive unaltered forms of the environmental sounds that it would naturally perceive.

Subjecting the individual to altered environmental sounds may include delivering at least a portion of the first audio sample to the right ear of the person experiencing tinnitus or delivering at least a portion of the second audio sample to the left ear of the person experiencing tinnitus. In this manner, the individual will be subjected to receive a transposed version of the left and/or right environmental sounds that the individual would naturally perceive. The portions delivered may be the substantially full portions of the acquired audio sounds.

The method step 104 of subjecting the individual to altered environmental sounds may include blocking the individual from perceiving at least a portion of the environmental sounds that the individual would naturally perceive. This blocking may be accomplished by any suitable noise cancelation technique. For instance, ear defenders or other natural sound blocking devices may be used. By blocking a substantial portion of the sound, the individual may essentially experience only the altered environmental sounds provided. This may help to ensure an increase in the perceptual multisensory error signals in the brain of the individual.

In addition to a spatial alteration, the altered environmental sounds provided in the method 100 may include at least one non-spatial alteration to the environmental sounds that the individual would naturally perceive. As one example, the altered environmental sounds may include an amplitude alteration to at least a portion of the environmental sounds that the individual would naturally perceive. For instance, the amplitude of the environmental sounds that the individual would naturally perceive may be increased or decreased in order to further induce multisensory error signals in the brain of the individual. Likewise, the altered environmental sounds may include a frequency alteration to at least a portion of the environmental sounds that the individual would naturally perceive. For instance, the altered environmental sounds may be specifically limited to frequency band. The frequency alteration to the environmental sounds may limit the frequency range of the altered environmental sounds to one or more narrow band(s) surrounding one or more symptomatic frequencies experienced by the individual. As an example, an individual suffering a symptomatic frequency of tinnitus in a left ear may be provided spatially altered environmental sounds to only the left ear and only in a range surrounding a symptomatic frequency. By spatially altering only the frequencies close to the tinnitus experienced by the individual, this may allow for a more tailored treatment that may be more tolerable for the individual. The altered environmental sounds may be limited to a range of within 1000 Hz, within 100 Hz, or within 50 Hz of a symptomatic frequency experienced by the individual. Alternatively, the altered environmental sounds may be limited to a range of within about one octave or within about a half octave of a symptomatic frequency experienced by the individual. The symptomatic frequencies for each individual may be predetermined using any suitable method. This targeted approach may teach the brain of the individual that only the signals in the provided range are unreliable and should be disregarded.

The at least one non-spatial alteration to the altered environmental sounds may include a time delay between when the individual is subjected to the altered environmental sounds and when the individual would otherwise naturally perceive an unaltered form of the sounds. The time delay may specifically be at least 10 milliseconds, at least 20 milliseconds, at least 40 milliseconds, at least 70 milliseconds, or at least 100 milliseconds. In particular, a time delay of between about 40 milliseconds and about 80 milliseconds have been shown to be perceived as being annoying without the listener or viewer recognizing the time delay. Consequently, this range may be suitable to induce a moderate error signal that is not obvious to the individual. Similar to the frequency and amplitude alterations discussed above, this additional modification may introduce an error in the soundscape that beneficially indicates to the brain of the individual that the sounds are unreliable and thus should be toned down, ignored, or re-calibrated. Any number of additional non-spatial alterations may be performed on the environmental sounds that the individual would naturally perceive. By specifically introducing additional error signals, the effectiveness of the treatment may be improved. As one example, the method may include subjecting the individual to altered environmental sounds that include a spatial alteration in addition to both a frequency alteration and a time delay. For instance, an afflicted ear of an individual may be specifically subjected to a time-delayed, high-pitch frequency range of environmental sounds acquired proximate to the other ear of the individual.

The method step 104 of subjecting an individual to altered environmental sounds may last for a fixed, predetermined amount of time. Alternatively, the individual may be subjected to the altered environmental sounds for a minimum period of time. For instance, the individual may be subjected to the altered environmental sounds for a time period of at least 20 minutes, at least 30 minutes, at least 60 minutes, at least 90 minutes, or at least 120 minutes. The method step 104 of subjecting the individual to altered environmental sounds may be repeated. As one example, the individual may be subjected to the altered environmental sounds for at least 30 minutes, at least 60 minutes, at least 90 minutes, or at least 120 minutes every day for a period of at least 1 week, at least 2 weeks, at least 4 weeks, at least 10 weeks, at least one year, or indefinitely. As another example, the individual may be subjected to short periods of treatment, such as a treatment for a period of about 1 second, about 3 seconds, about 5 seconds, or about 10 seconds every minute or every other minute. In order to avoid adaptation to the treatment and reduced effectiveness, the environmental sounds may be intermittently repeated for a series of predetermined, varying periods of time. The length and period of treatment may specifically be applied until the symptoms experienced by the individual have been substantially reduced or no longer persist.

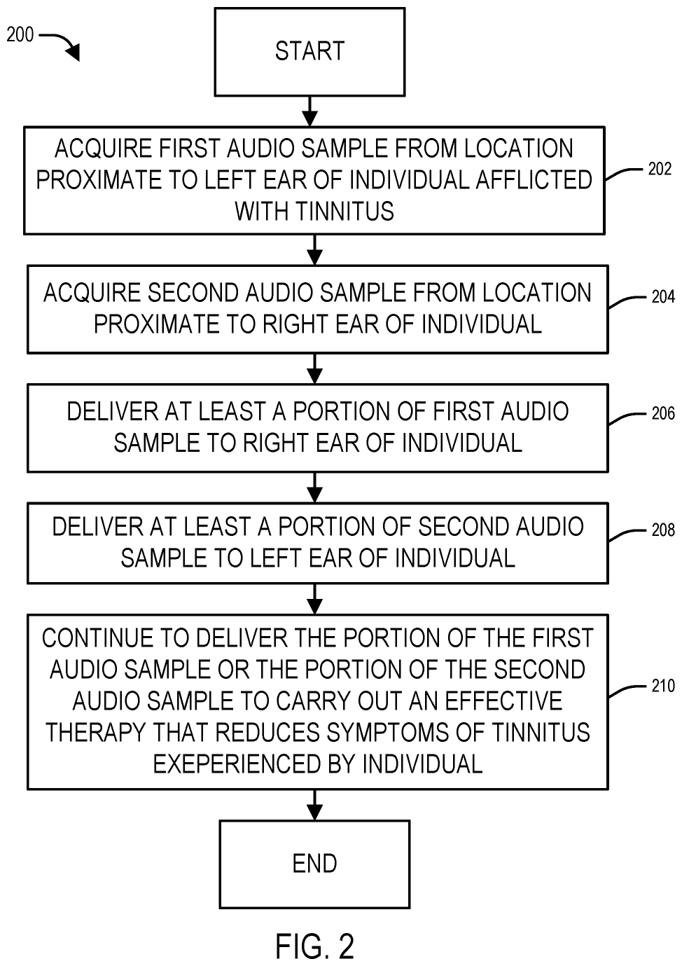

Referring to FIG. 2, a flowchart of another exemplary method of treating tinnitus 200 is provided. The method 200 includes a first step method 202 of acquiring a first audio sample from a location proximate to a left ear of an individual afflicted with tinnitus, a second step 204 of acquiring a second audio sample from a location proximate to a right ear of the individual, a third step 206 of delivering at least a portion of the first audio sample to the right ear of the individual, a fourth step 208 of delivering at least a portion of the second audio sample to the left ear of the individual, and a fifth step 210 of continuing delivery of the portion of the first audio sample delivered to the right ear of the individual or the portion of the second audio sample delivered to the left ear of the individual to carry out an effective therapy that reduces symptoms of tinnitus experienced by the individual. The method 200 may utilize and include any of the relevant teachings disclosed above for the method 100.

A reduction of symptoms of tinnitus experienced by the individual may include a reduction in a measured Tinnitus Handicap Inventory (THI) score, a Tinnitus Reaction Questionnaire (TRQ) score, or a Tinnitus Handicap Questionnaire (THQ) score of the individual. For instance, an effective therapy may be considered to have occurred after a reduction in the THI score of the individual by at least 6 points, at least 8 points, at least 10 points, at least 15 points, or at least 25 points. One of skill in the art will recognize that other suitable tinnitus handicap evaluation methods can be readily substituted for the THI score, TRQ score, or the THQ score.

Similar to the method 100 described above, the method 200 may further include blocking the individual from perceiving at least a portion of the environmental sounds that the individual would naturally perceive. Again, this blocking step may occur actively (e.g. using active noise cancellation) or passively (e.g. using a noise blocking apparatus such as an ear defender).

Also similar to method 100, the method 200 may include an additional step of modifying the first audio sample or the second audio sample to include at least one non-spatial alteration prior to delivery to the individual. Again the non-spatial alteration may be an amplitude alteration, a frequency alteration, a time delay, or any combination thereof.

The method step 210 of continuing delivery of the portion of the first audio sample or the portion of the second audio sample may include repeating the delivery of the portion of the first audio sample or the portion of the second audio sample at least once. Continuing delivery of the portion of the first audio sample or the portion of the second audio sample may include intermittently repeating the delivery of the portion of the first audio sample or the portion of the second audio sample for a series of predetermined, varying periods of time. For instance, continuing delivery of the portion of the first audio sample or the portion of the second audio sample may include repeating the delivery of the portion of the first audio sample or the portion of the second audio sample for at least 30 minutes, at least 60 minutes, at least 90 minutes, or at least 120 minutes every day for a period of at least 1 week, at least 2 weeks, at least 4 weeks, at least 10 weeks, at least one year, or indefinitely.

In one aspect, systems for delivering a therapy for tinnitus are provided. In order to ensure that an effective therapy treatment is provided in accordance with the techniques described herein, the system may utilize any steps or variations of the methods 100 and 200 described above.

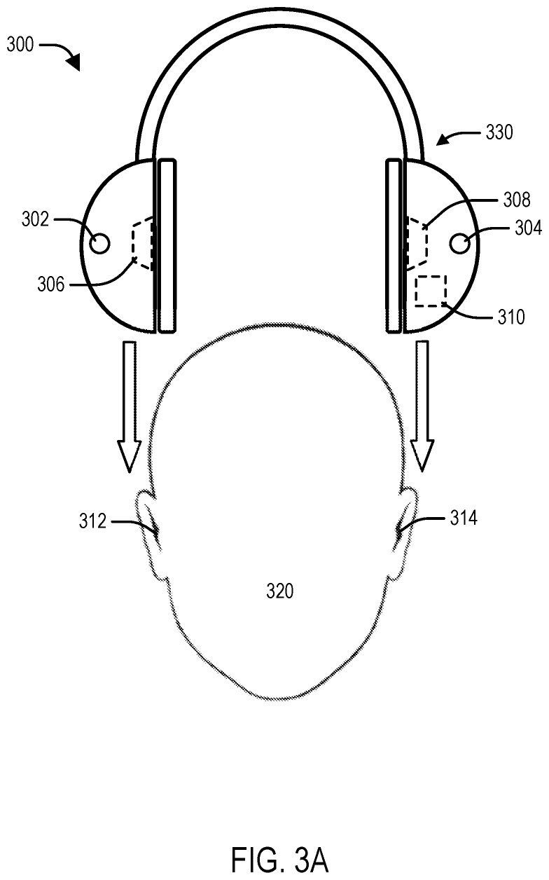

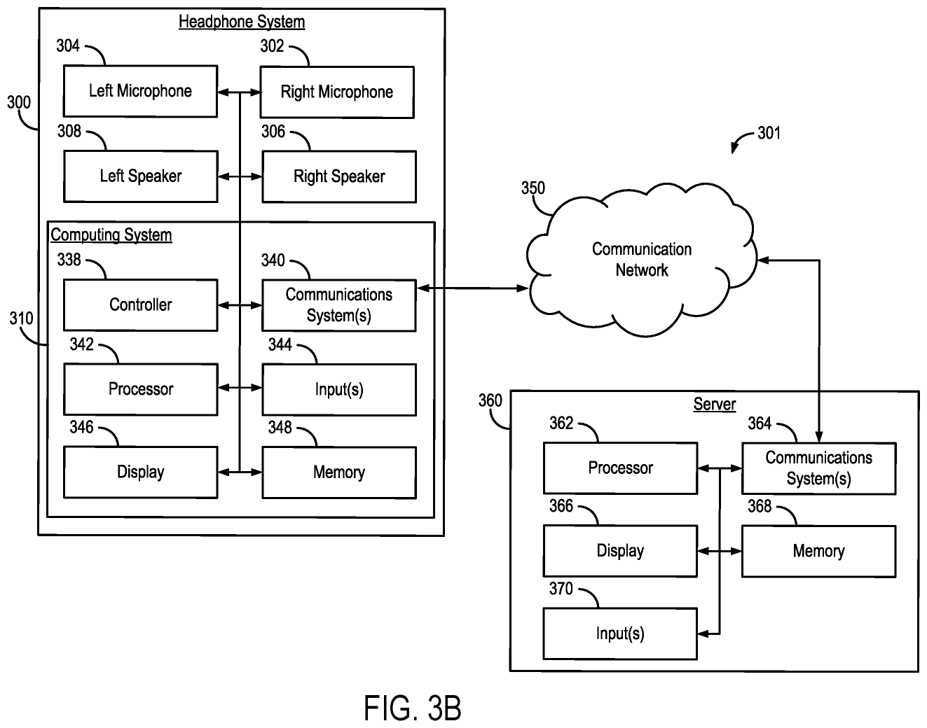

Referring to FIG. 3A, an exemplary system 300 for delivering a therapy for tinnitus is depicted. The system 300 includes a right microphone 302 configured to sample environmental sounds from a location proximate to a right ear 312 of an individual 320 afflicted with tinnitus, and a left microphone 304 configured to sample environmental sounds from a location proximate to a left ear 314 of the individual 320. The system 300 also includes a right speaker 306 configured to transmit sound to the right ear 312 of the individual 320 and a left speaker 308 configured to transmit sound to the left ear 314 of the individual 320. The system also includes a computing system 310 housing a controller configured to selectively control the right speaker 306 to deliver the environmental sounds sampled by the left microphone 304 to the right ear 312 of the individual 320 and selectively control the left speaker 308 to deliver the environmental sounds sampled by the right microphone 302 to the left ear 314 of the individual 320, in order to reduce symptoms of tinnitus experienced by the individual 320. In this exemplary system 300, all components are housed within a headphone system 330, which is an over-ear headphone unit configured to be positioned onto the head of the individual as shown by the depicted arrows. However, it should be appreciated that numerous other headphones system configurations may be used for the system, including alternative headphone constructions or hearing aid devices, such as in-ear hearing aids.

Referring to FIG. 3B, a schematic diagram 301 of the system 300 interacting with a remote server 360 is shown. The system 300 can receive one or more types of data including treatment data, such as data including information on the time, frequency, and type of treatment sound provided to the individual, and symptomatic data, such as the time, frequency, and extent of symptoms experienced by the individual. As shown, the system 300 can communicate information about data received to a remote server 360 over a communication network 350, which can be accessed by any authorized individuals or medical practitioners. In this manner, treatment and symptomatic data acquired by the system 300 may be used to evaluate patient progress and customize future treatment regimens.

The computing system 310 may be integrated with the headphone system 300, and may optionally house and provide electrical communication between the controller 338, a communications system(s) 340, a processor 342, input(s) 344, a display 346, and a memory 348. These components may allow the system 300 to measure and store treatment and symptom data and to directly provide such data to the remote server 360 via the communication network 350.

The communication network 350 can be any suitable communication network or combination of communication networks. For example, the communication network 350 can include a Wi-Fi network (which can include one or more wireless routers, one or more switches, etc.), a peer-to-peer network (e.g., a Bluetooth network), a cellular network (e.g., a 4G network, a 5G network, etc., complying with any suitable standard, such as CDMA, GSM, LTE, LTE Advanced, WiMAX, etc.), a wired network, and so on. In some forms, the communication network 350 can be a local area network, a wide area network, a public network (e.g., the Internet), a private or semi-private network (e.g., a corporate or university intranet), any other suitable type of network, or any suitable combination of networks.

The processor 342 can be any suitable hardware processor or combination of processors, such as a central processing unit (“CPU”), a graphics processing unit (“GPU”), and so on. The processor 342 may be further configured to control the left microphone, right microphone, or display to provide information to the individual, such as to prompt the individual to input symptom data. The processor 342 may modify various treatment aspects based on inputs it receives. For instance, the processor 342 may be configured to determine a speed of the individual and stop providing altered environmental sounds sound if a threshold speed is exceeded, in order to maintain the safety of the individual.

The computing system may be further configured to communicate input symptom data over time or other information to the individual via either the display 346, or the left microphone 304, or the right microphone 306. The display 346 can include any suitable display devices, such as a small screen or one or more informational light sources. The inputs 344 may include any suitable input devices and/or sensors that can be used to receive user input from the individual, such as one or more buttons, switches, or dials. In some forms, the left microphone 304 and right microphone 302 may be the only inputs.

The communications systems 340 can include any suitable hardware, firmware, and/or software for communicating information over the communication network 350 and/or any other suitable communication networks. For example, the communications systems 340 can include one or more transceivers, one or more communication chips and/or chip sets, and so on. For instance, the communications systems 340 can include hardware, firmware and/or software that can be used to establish a Wi-Fi connection, a Bluetooth connection, a cellular connection, an Ethernet connection, and so on.

Using the memory 348, the computing system may be configured to receive and store treatment data, wherein treatment data includes temporal information on the delivery of the environmental sounds by the right speaker and the left speaker. For instance, the computing system may be further configured to store at least one of a duration or an audio level of the portion of the first audio sample delivered to the right ear of the person experiencing tinnitus or the portion of the second audio sample delivered to the left ear of the person experiencing tinnitus. The computing system may be further configured receive and store symptom data from the individual. In some forms, the memory 348 can include any suitable storage device or devices that can be used to store instructions, values, data, or the like, that can be used, for example, by the processor 342 to present content using the display 346, to communicate with the server 360 via the communications system(s) 340, and to modify the applied treatment through the left speaker 308 and the right speaker 306. The memory 348 can include any suitable volatile memory, non-volatile memory, storage, or any suitable combination thereof. For example, the memory 348 can include RAM, ROM, EEPROM, one or more flash drives, one or more hard disks, one or more solid state drives, one or more optical drives, and so on. The memory 348 may have encoded thereon, or otherwise stored therein, a computer program for controlling operation of the system 300. For instance, the processor 342 may execute at least a portion of a computer program to modify speaker output, receive content from the server 360, transmit information to the server 360, and so on.

The server 360 may include a processor 362, a display 366, one or more inputs 370, one or more communications systems 364, and/or a memory 368. The processor 362 can be any suitable hardware processor or combination of processors, such as a CPU, a GPU, and so on. The display 366 can include any suitable display devices, such as a computer monitor, a touchscreen, a television, and so on. The inputs 370 can include any suitable input devices and/or sensors that can be used to receive user input, such as a keyboard, a mouse, a touchscreen, a microphone, and so on.

The communications systems 364 can include any suitable hardware, firmware, and/or software for communicating information over the communication network 350 and/or any other suitable communication networks. For example, the communications systems 364 can include one or more transceivers, one or more communication chips and/or chip sets, and so on. For instance, the communications systems 364 can include hardware, firmware and/or software that can be used to establish a Wi-Fi connection, a Bluetooth connection, a cellular connection, an Ethernet connection, and so on.

In some embodiments, the memory 368 can include any suitable storage device or devices that can be used to store instructions, values, data, or the like, that can be used, for example, by the processor 362 to present content using the display 366, to communicate with one or more systems, and so on. The memory 368 can include any suitable volatile memory, non-volatile memory, storage, or any suitable combination thereof. For example, the memory 368 can include RAM, ROM, EEPROM, one or more flash drives, one or more hard disks, one or more solid state drives, one or more optical drives, and so on. In one form, the memory 368 can have encoded thereon a server program for controlling operation of the server 360. For instance, the processor 412 can execute at least a portion of the server program to transmit information and/or content (e.g., data, images, a user interface) to the system 300, receive information and/or content from the system 300, receive instructions from one or more devices (e.g., a personal computer, a laptop computer, a tablet computer, a smartphone), and so on.

Any suitable computer readable media can be used for storing instructions for performing the functions and/or processes described herein. As one example, a non-transitory, computer-readable storage medium having instructions stored thereon that, when executed by a processor, cause the processor to carry out steps including controlling a right microphone to sample environmental sounds from a location proximate to a right ear of an individual afflicted with tinnitus, controlling a left microphone to sample environmental sounds from a location proximate to a left ear of the individual, and selectively controlling a right speaker configured to be coupled to the right ear of the individual to deliver the environmental sounds sampled by the left microphone to the right ear or selectively control a left speaker configured to be coupled to the left ear of the individual to deliver the environmental sounds sampled by the right microphone to the left ear to reduce symptoms of tinnitus experienced by the individual is provided.

In some aspects, the computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

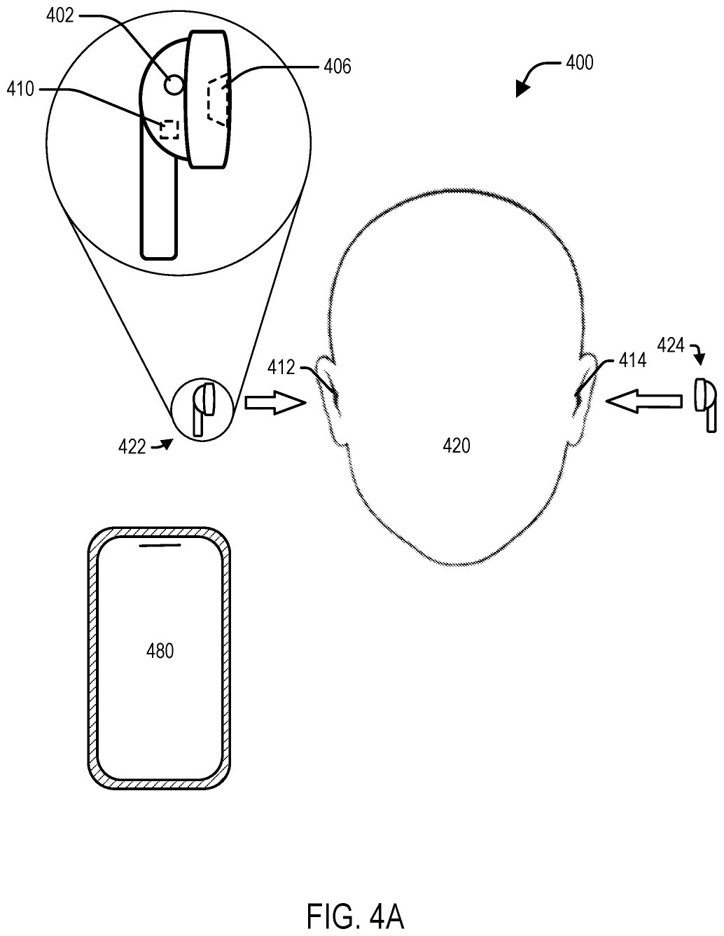

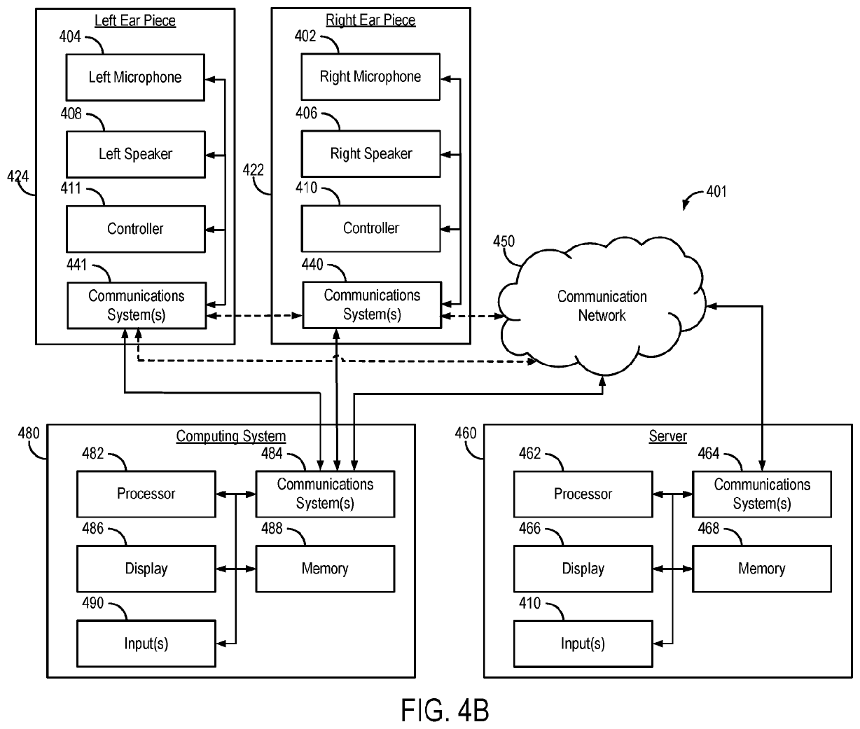

Referring to FIG. 4A, an exemplary system 400 for delivering a therapy for tinnitus is depicted. The system 400 includes a right ear piece 422 housing a right microphone 402 configured to sample environmental sounds from a location proximate to a right ear 412 of an individual 420 afflicted with tinnitus. The right ear piece 422 also includes a right speaker 406 configured to transmit sound to the right ear 412 of the individual 420. The system 400 further includes a left ear piece 424 housing a left microphone (not depicted) configured to sample environmental sounds from a location proximate to a left ear 414 of the individual 420. The left ear piece 424 also has a left speaker (not depicted) configured to transmit sound to the left ear 414 of the individual 420. As shown, the right ear piece has a controller 410 configured to selectively control the right speaker 406 to deliver the environmental sounds sampled by the left microphone (not depicted) to the right ear 412 of the individual 420 to reduce symptoms of tinnitus experienced by the individual 420. Likewise, the left ear piece has its own controller configured to selectively control the left speaker to deliver the environmental sounds sampled by the right microphone 402 to the left ear 414 of the individual 420 to reduce symptoms of tinnitus experienced by the individual 420. Both the right ear piece 422 and the left ear piece 424 include communication systems which allow for electrical communication between each controller and a remote computing system 480. The computing system 480 may control or modify the treatment therapy being issued by the left ear piece 424 and the right ear piece 412. Furthermore, the computing system may store, display, and/or communicate treatment, symptom, or other system data, and may provide notifications to the individual 420 regarding the provided treatment. As shown, the computing system 480 may be a smartphone or tablet device.

Referring to FIG. 4B, a schematic diagram 401 of the system 400 interacting with a remote server 460 is shown. Each of the left ear piece 424, the right ear piece 422, and the computing system 480 can receive one or more types of data including treatment data, such as data including information on the time, frequency, and type of treatment sound provided to the individual, and symptomatic data, such as the time, frequency, and extent of symptoms experienced by the individual. As shown, the computing system 480 can communicate information about data received to a remote server 460 over a communication network 450, which can be accessed by any authorized individuals or medical practitioners. In this manner, treatment and symptomatic data acquired by the system 400 may be used to evaluate patient progress and customize future treatment regimens. In some aspects, instead of relying on the computing system 480, the left ear piece 424 and/or the right ear piece 422 may communicate directly with each other and with the communication network 450 to provide data to the server 460.

Similar to the computing system integrated with the headphone system of FIGS. 3A-3B, the remote computing system 480 may optionally house and provide electrical communication between the controller 338, a communications system(s) 340, a processor 342, a input(s) 344, a display 346, and a memory 348, which may incorporate any of the teachings described above for each component, respectively. By providing the computing system 480 as a separate smartphone, tablet, or comparable device in electrical communication with the left ear piece 424 and the right ear piece 422, the depicted system may advantageously rely on simplified, low-cost headphone systems and computing devices that are already commercially available and used by many individuals. Utilizing the full display capabilities of commercially available smartphone, tablet, or comparable devices may also improve the quality and range of information provided to the user and the input information that the individual may provide to the system.

Similar to the communication network and server of FIG. 4B the communication network 450 can be any suitable communication network or combination of communication networks and the server 460 may include a processor 462, a display 466, one or more inputs 470, one or more communications systems 464, and/or a memory 468, which may incorporate any of the teachings described above for each component, respectively.

Although specific features of various embodiments of the disclosure may be shown in some drawings and not in others, this is for convenience only. In accordance with the principles of the disclosure, any feature of a drawing may be referenced and/or claimed in combination with any feature of any other drawing.

This written description uses examples to disclose the various aspects and embodiments, including the best mode, and also to enable any person skilled in the art to practice the embodiments, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

### EXAMPLES

The following Examples are provided in order to demonstrate and further illustrate certain embodiments and aspects of the present disclosure and are not to be construed as limiting the scope of the disclosure.

### Example 1

In one, non-limiting study, a device was constructed to evaluate the effectiveness of spatial sound transposition in a subject, also referred to as auditory mirror therapy, as a treatment for subjects afflicted with tinnitus.

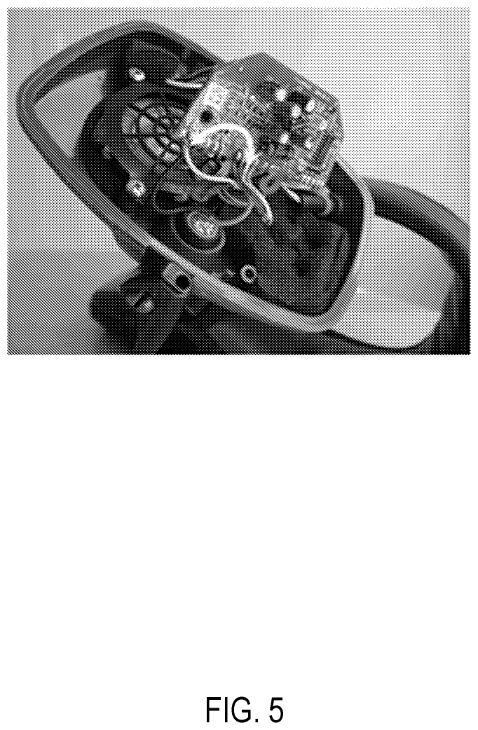

An auditory mirror therapy (AMT) device was built by modifying on ear defenders with built in sound amplification (Impact® Sport Howard Leight by Honeywell) Smithfield, RI, USA). Specifically, the connections of the left and right microphone tabs on the circuit board of the ear defender headphones were swapped, thereby transposing the left ear and right ear sounds. Based on initial reports that the device was not comfortable to wear for long durations, a second and more comfortable prototype was constructed. The second auditory mirror therapy device was a similarly modified ear defender (3M™ Peltor™ SV Tactical Pro Hearing Protector, fitted with PELTOR Camelback Gel Sealing Rings (St. Paul, MN, USA)). FIG. 5 depicts an image of the modified experimental 3M™ Peltor™ SV Tactical Pro Hearing Protector, where the arrows indicate the microphones wires which have been rewired to function as described herein.

Twenty subjects with chronic (>3 months), tonal (i.e. beep- or whistle-like) tinnitus were recruited from the community for the study. Exclusion criteria included significant medical history, alcohol or substance abuse, and current or past history of balance-, vertigo- and/or vestibular-symptoms including Meniere's disease. The 20 participants were enrolled and baseline tinnitus awareness, intensity, Tinnitus Reaction Questionnaire (TRQ), Tinnitus Handicap Inventory (THI), and anxiety ratings were collected for each subject. Participants reported on average 17 (+14, range 1 to 50) years of tinnitus.

During the study, participants used the AMT device for 14 days at home for up to 2 hours per day. After two weeks of use, subjects completed the tinnitus questionnaires and returned the device. 18 participants (8 men, 10 women, mean (±SD) age 60 (±8) years) completed the full study. Subjects who completed the study wore the AMT device between 11 and 75 hours, (average 36 (+19) hours).

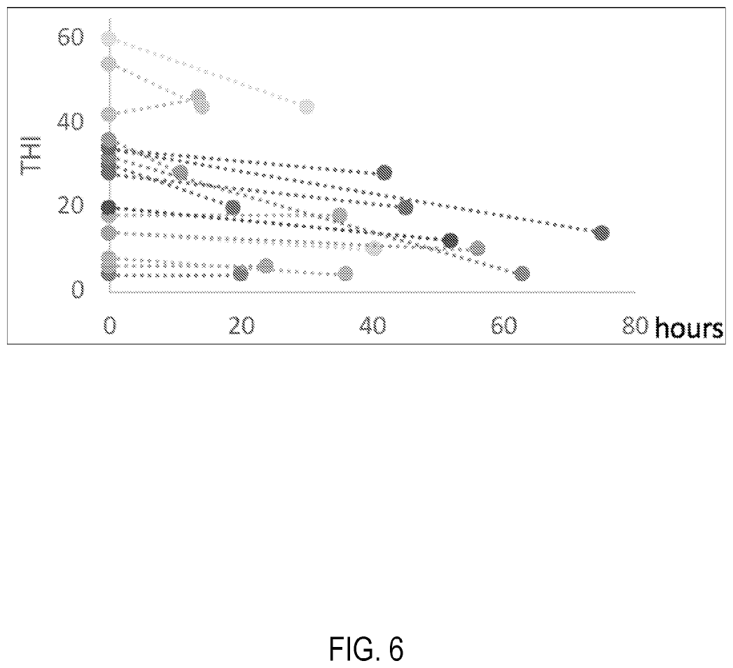

FIG. 6 depicts the experimental results from the study, charting each participants THI value over the number of hours the device was used. As shown, at the completion of the intervention, there was a significant reduction in tinnitus awareness (−35%, p=0.00057) and in THQ (−24%, p=0.0014). Trend reductions, not significant after Bonferroni correction, were observed in TRQ (−24%, p=0.018 and loudness at worst (−8%, p=0.049) were observed. As shown in FIG. 6, there was a significant dose-response relationship between the number of hours the device was used and the change in THI, Pearson r=0.70, p=0.0026.

The most common adverse effect reported was that the AMT device was tight and uncomfortable, sometimes inducing headache after prolonged use. The two subjects that did not complete the study indicated that their tinnitus got worse by wearing the device, and discontinued use after 3 and 6 hours.

In conclusion, treating the subjects with spatially altered environmental sounds using the AMT technique led to significant reductions in both tinnitus awareness and handicap. A clinically relevant improvement in THI ratings (greater than six points 24) was noted in 56% of subjects. The results support the finding that exposing the audio-visual integration system to a major challenge can reduce tinnitus awareness and decrease perceived handicap of tinnitus. Without being bound by theory, it is contemplated that this improvement may occur either through reducing the “filling in” mechanism or a change in the gain and attention paid to spontaneous fluctuations in deafferented or abhorrent primary auditory neurons. As there were no changes in state or trait anxiety levels, reduced stress did not appear to drive the observed changes. In the pilot study there was no control group, and audiological measures or tinnitus matching procedures were not obtained. Also, potential long-term benefits of the intervention were not studied.

The present invention has been described in terms of one or more preferred embodiments, and it should be appreciated that many equivalents, alternatives, variations, and modifications, aside from those expressly stated, are possible and within the scope of the invention.

1. A method of treating tinnitus comprising:
subjecting an individual afflicted with tinnitus to altered environmental sounds, wherein the altered environmental sounds are spatially altered forms of environmental sounds that the individual would naturally perceive.

2. The method of claim 1, wherein the individual has a right ear and a left ear and the method step of subjecting the individual to the altered environmental sounds includes acquiring a first audio sample from a location proximate to the left ear and acquiring a second audio sample proximate to the right ear.

3. The method of claim 2, wherein subjecting the individual to altered environmental sounds includes delivering at least a portion of the first audio sample to the right ear of the person experiencing tinnitus or delivering at least a portion of the second audio sample to the left ear of the person experiencing tinnitus.

4. The method of claim 1, wherein subjecting the individual to altered environmental sounds includes blocking the individual from perceiving at least a portion of the environmental sounds that the individual would naturally perceive.

5. The method of claim 1, wherein the altered environmental sounds include at least one non-spatial alteration to the environmental sounds that the individual would naturally perceive.

6. The method of claim 5, wherein the altered environmental sounds include an amplitude or frequency alteration or a time delay to at least a portion of the environmental sounds that the individual would naturally perceive.

7-14. (canceled)

15. A method of treating tinnitus comprising:
acquiring a first audio sample from a location proximate to a left ear of an individual afflicted with tinnitus;
acquiring a second audio sample from a location proximate to a right ear of the individual;
delivering at least a portion of the first audio sample to the right ear of the individual;
delivering at least a portion of the second audio sample to the left ear of the individual; and
continuing delivery of the portion of the first audio sample delivered to the right ear of the individual or the portion of the second audio sample delivered to the left ear of the individual to carry out an effective therapy that reduces symptoms of tinnitus experienced by the individual.

16. The method of claim 15, wherein a reduction of symptoms of tinnitus experienced by the individual includes a reduction in a measured Tinnitus Handicap Inventory (THI) score of the individual.

17. The method of claim 15 comprising:
blocking the individual from perceiving at least a portion of the environmental sounds that the individual would naturally perceive.

18. The method of claim 15 comprising:
modifying the first audio sample or the second audio sample to include at least one non-spatial alteration prior to delivery to the individual.

19. The method of claim 18, wherein the first audio sample or the second audio sample is modified to include at least one of an amplitude alteration, a frequency alteration, or a time delay.

20-22. (canceled)

23. The method of claim 15, wherein the method step of continuing delivery of the portion of the first audio sample or the portion of the second audio sample includes repeating the delivery of the portion of the first audio sample or the portion of the second audio sample at least once.

24. The method of claim 15, wherein the method step of continuing delivery of the portion of the first audio sample or the portion of the second audio sample includes intermittently repeating the delivery of the portion of the first audio sample or the portion of the second audio sample for a series of predetermined, varying periods of time.

25. The method of claim 15, wherein the method step of continuing delivery of the portion of the first audio sample or the portion of the second audio sample includes repeating the delivery of the portion of the first audio sample or the portion of the second audio sample for at least 30 minutes, at least 60 minutes, at least 90 minutes, or at least 120 minutes every day for a period of at least 1 week, at least 2 weeks, at least 4 weeks, at least 10 weeks, at least one year, or indefinitely.

26. A system for delivering a therapy for tinnitus, the system comprising:
a right microphone configured to sample environmental sounds from a location proximate to a right ear of an individual afflicted with tinnitus;
a left microphone configured to sample environmental sounds from a location proximate to a left ear of the individual,
a right speaker configured to transmit sound to the right ear of the individual;
a left speaker configured to transmit sound to the left ear of the individual; and
a controller configured to selectively control the right speaker to deliver the environmental sounds sampled by the left microphone to the right ear of the individual and selectively control the left speaker to deliver the environmental sounds sampled by the right microphone to the left ear of the individual to reduce symptoms of tinnitus experienced by the individual.

27. The system of claim 26 further comprising a computing system in electrical communication with the controller, the computing system configured to receive an input from the individual and instruct the controller to selectively control the right speaker and the left speaker.

28. The method of claim 27, wherein the computing system is integrated with a headphone device comprising at least one of the right speaker and right microphone, or the left speaker and left microphone.

29. (canceled)

30. The method of claim 27, wherein the computing system is configured to receive and store treatment data, wherein treatment data includes temporal information on the delivery of the environmental sounds by the right speaker and the left speaker.

31. The method of claim 27, wherein the computing system is further configured receive and store symptom data from the individual.

32-33. (canceled)

34. The method of claim 31, wherein the computing system is further configured to provide treatment data or symptom data to a server accessible to a remote healthcare facility.

35. The method of claim 27, wherein the computing system is further configured to determine a speed of the individual and stop providing altered environmental sounds sound if a threshold speed is exceeded.

36. The method of claim 27, wherein the computing system is further configured to store at least one of a duration or an audio level of the portion of the first audio sample delivered to the right ear of the person experiencing tinnitus or the portion of the second audio sample delivered to the left ear of the person experiencing tinnitus.

37. A non-transitory, computer-readable storage medium having instructions stored thereon that, when executed by a processor, cause the processor to carry out steps comprising:
controlling a right microphone to sample environmental sounds from a location proximate to a right ear of an individual afflicted with tinnitus;
controlling a left microphone to sample environmental sounds from a location proximate to a left ear of the individual,
selectively controlling a right speaker configured to be coupled to the right ear of the individual to deliver the environmental sounds sampled by the left microphone to the right ear or selectively controlling a left speaker configured to be coupled to the left ear of the individual to deliver the environmental sounds sampled by the right microphone to the left ear to reduce symptoms of tinnitus experienced by the individual.
